(19) United States
(12) Patent Application Publication
(10) Pub. No.: US 2007/0167418 A1
(43) Pub. Date: Jul. 19, 2007

(54) Progesterone/testosterone cream for erectile dysfunction

(76) Inventor: Steven Wallace Ferguson, Powellsville, NC (US)

Correspondence Address: STEVEN W. FERGUSON, MD, CMD, P. O. BOX 69, 105 COMMERCE STREET, POWELLSVILLE, NC, 27967, US

(21) Appl. No.: 11/221,652

(22) Filed: Sep. 7, 2005

Related U.S. Application Data
(60) Provisional application No. 60/522,259, filed on Sep. 7, 2004.

(51) Int. Cl. A61K 31/57

(52) U.S. Cl. 514/170

(57) ABSTRACT

We have found a consistent pattern of hormonal abnormalities in patients with ERECTILE DYSFUNCTION. Our study group included men from age 21 to 88 years old. By helping to correct the hormonal imbalance of progesterone and testosterone towards normal values, most of the men in our study were able to obtain normal erectile function. By using a progesterone/testosterone cream we were able to help correct the hormonal imbalance commonly found in men with ERECTILE DYSFUNCTION.

### CROSS REFERENCE TO RELATED INFORMATION

This application provides priority over provisional application 60/522,259 filed out Sep. 7, 2004.

### STATEMENT REGARDING FEDERALLY SUPPORTED RESEARCH OR DEVELOPMENT

N/A

### BACKGROUND OF THE INVENTION

Erectile dysfunction is one of the most common chronic disorders affecting men and becomes increasingly prevalent with age. Data from the Massachusetts Male Aging study showed that 52% of men aged 40 to 70 years reported some degree of ED. A similar prevalence of ED has also been found in numerous countries worldwide, affecting greater than 40% of men older than 60 years of age in Finland, Italy, Japan, the United Kingdom, Australia and Iran.

My study was designed to evaluate the effect of bio-identical progesterone/testosterone on the erectile dysfunction of men. The study was performed over a period of two years. Twenty patients were randomly involved in this clinical study ranging in ages from twenty-one to eighty-eight years. The men had a multiplicity of medical conditions including one or more of the following disease processes: hypertension, BPH, diabetes, elevated cholesterol, cardiovascular disease, lung or prostate cancer, cerebral palsy, COPD, renal insufficiency and depression. At the time this study was undertaken the initial goal was to improve erectile dysfunction regardless of the preexisting medical problems. At first, I attempted to distinguish between organic and psychogenic erectile dysfunction. I attempted to optimize their medical management and simply added our product to there daily regiment without altering any other variables.

My formulation is a Bio-Identical Progesterone-Testosterone compound in a cream or gel. The men in the study received a small amount of this mixture daily. They used one to three pea sized allocates of cream, to their inner arms, chest, face, or neck.

### BRIEF SUMMARY OF INVENTION

A nutriceutical alternative to medically treated ERECTILE DYSFUNCTION: we have found a consistent pattern of hormonal abnormalities in patients with ERECTILE DYSFUNCTION. Our study group included men from age 21 to 88 years old. We obtained informed consent and confidentiality statements from each of the men and pharmacist involved in our study. We used a bio-identical testosterone and progesterone powder in a mixture ranging from 0.5 to 5% progesterone and 2 to 20% testosterone dissolved in a small amount of solvent (Olive Oil, Mineral Oil, Eucerin Cream, Aquapha or similar Cream). This solution was then applied to the skin areas without hair (inner arm, chest, neck, face, etc.) once a day. In this study, the men had multiple medical problems to include hypertension, BPH, diabetes, elevated cholesterol, cardiovascular disease, cerebral palsy, BOPD, renal insufficiency, and depression. We did however attempt to distinguish between organic and psychogenic ERECTILE DYSFUNCTION. We attempted to eliminate any patients with psychogenic ERECTILE DYSFUNCTION and to optimize their medical care for their different medical conditions.

We had tremendous success in correcting ERECTILE DYSFUNCTION despite the number of co-existing medical problems. In our study ERECTILE DYSFUNCTION showed significant improvement in as little as three weeks and as long as six months with an average being two to three months.

### DETAILED DESCRIPTION OF INVENTION

Below you will find supportive documentation of Progesterone/Testosterone Cream in helping to improve ERECTILE DYSFUNCTION.

### BACKGROUND INFORMATION

### Sections of the JAMA Article With My Comments in Red

Morgentaler, Abraham, M. D. “A 66 Year Old Man with Sexual Dysfunction.” JAMA Magazine. Vol. 291, 24. 23/30 Jun. 2004

The classification of Male Sexual Dysfunction grossly includes the following:
Erectile dysfunction—organic/psychogenic
Hypoactive desire—diminished or absent libido
Ejaculatory dysfunction i.e. premature, difficulty achieving orgasm including, Anorgasmia Anatomical abnormalities, eg, Peyronic disease

Sexual dysfunction can lead to depression and profoundly altered sense of self-esteem that negatively affects many relationships; increased awareness and treatment are thus to be greatly encouraged, due to the profound benefits in life satisfaction that may result.¹

Erectile dysfunction is one of the most common chronic disorders affecting men and becomes increasingly prevalent with age. Data from the Massachusetts Male Aging study showed that 52% of men aged 40 to 70 years reported some degree of ED.⁴ A similar prevalence of ED has also been found in numerous countries worldwide, affecting greater than 40% of men older than 60 years of age in Finland,⁵ Italy,⁶ Japan,⁷ the United Kigdom,⁸ Australia,⁹ and Iran. ¹⁰

Risk factors for ED include age, diabetes mellitus, hypertension, hyperlipidaemia, coronary and peripheral vascular disease, smoking, obstructive voiding symptoms, obesity, renal failure and alcoholism.^{4,11} (Our study was not able evaluate the extent of erectile dysfunction in relationship to the above. But most of our men have had success notwithstanding their medical problems.)

Medications are also a common contributing factor. The most common offenders include antihypertensive medications, digoxin, antidepressants, spironolactone adrenergic agents and testosterone-lowering medications such as gonadotropin releasing hormone agonist/antagonists. New onset Ed associated with a new medication or an increased dosage suggests medication as the likely cause. For instance, the B-blocker class of antihypertensives has generally been considered one of the most common causes of medication-induced ED.¹² However, in a study of ninety-six men with newly diagnosed cardiovascular disease and without ED, 31% reported ED after beginning treatment with atenolol (50 mg) and being informed of its sexual adverse effects. In contrast, only 3% of men who were similarly treated reported ED when they were blinded as to the study drug.¹³ (In my study attempts were always made to optimize medical therapist and to decrease or remove excess medication whenever possible.).

PATHOPHYSIOLOGY OF ED: Corporal smooth muscle relaxation is mediated by the conversion of guanosine triphosphate (GTP) to cyclic guanine monophosphate (cGMP), under the influence of nitric oxide.^{17,18} The medications sildenafil, vardenafil, and tadalafil act by inhibiting the metabolism of CGMP by PDE 5, which is found almost exclusively in the corpora cavernosa.¹⁹

Erections may fail due to inadequate psychic arousal (eg. anxiety, depression); inadequate hormonal priming of sexual centers in the brain (eg. low testosterone); inadequate nerve signaling the penile vessels (eg, spinal cord injury, multiple sclerosis, radical prostatectomy); arterial insufficient (eg, atherosclerosis, vascular surgery, pelvic/perineal trauma); or impaired veno-occlusive ability within the corpora cavernosa (eg, radiation, Peyronic disease, atherosclerosis).¹

The diagnosis of ED is made by history alone and is defined by the inability to achieve or maintain an adequate erection for satisfactory sexual function.¹ If ED is present, it is useful to try to determine whether the problem is likely to be organic (physical) or psychological in etiology, since this may influence treatment.

The intermittent or sudden inability to have a firm erection suggests a psychogenic etiology. Low sexual desire suggests the diagnosis of hypogonadism, depression or a medication effect. Inability to maintain an erection is most often due to poor veno-occlusive function of the penis, but it is helpful to ask whether softening of the penis happens before or after orgasm, since men with premature ejaculation may describe their symptoms similarly.¹

PHYSICAL EXAMINATION. (A physical examination was performed on all of the patients in my study. Specific items to be evaluated include assessment of general health, vigor, mood and blood pressure. ) Testicular size and consistency should be noted, since small, soft testicles are associated with low serum testosterone level. Peripheral pulses should be evaluated. Neurological assessment should include a digital rectal examination (DRE), since nerve roots S2-4 mediate both erection and anal tone. The prostate should be assessed for size and for the presence of nodularity or asymmetry. (None of the men in my study were found to have any genitalia abnormalities.)

Ideally diagnostic test should be performed in your evaluation of erectile dysfunction: hematocrit, glucose, total and free testosterone, prolactin and lipid profile. Tests of thyroid function and hemoglobin A1c are optimal. The luteinizing hormone level should be measured if the history suggests hypogonadism. (In my study many of these diagnostic tests were performed but not consistently for all of the men.) Men with penile curvature or premature ejaculation do not require diagnostic testing but referral to urologist.

Total testosterone has been used most frequently; however, since the majority of circulating testosterone is bond tightly to sex hormone-binding globulin and is not biologically functional, exclusive reliance on the total testosterone assay will result in under-diagnosis of hypogonadism. The measurement of bioavailability or free testosterone appears to be much more useful. There is no basis for the use of age-adjusted reference values for testosterone, since men of any age will experience similar symptoms at low testosterone levels. More sophisticated tests, such as nocturnal penile tumescence and rigidity monitoring^{25,26} or penile Doppler ultrasound of the cavernosal arteries²⁷ can provide additional functional information but we did not include this in our study.

Pharmological Treatment

Sildenafil and vardenafil have similar pharmacokinetic properties, with peak serum concentrations at approximately one hour and a half-life of four to five hours.^{3,28} Tadalafil has a considerably longer half-life of approximately eighteen hours, with evidence that erectile function continues to be enhanced for at least thirty-six hours.²⁹ For example, in a double-blind randomized study of 532 men, successful intercourse was achieved in sixty-nine percent of men receiving 100 mg of sildernafil compared with twenty-two percent of men receiving placebo.³ Patient education is critical for optimal response to sildenafil. This includes informing the patient to take the medication on an empty stomach and to time sexual activity so that it occurs within one to six hours. In a study of 348 men using tadalafil (20 mg), fifty-nine percent successfully reported intercourses at thirty-six hours, compared with twenty-eight percent in the placebo group.³⁰ And in a multi-center, double-blind, placebo-controlled trial, sixty-nine percent of men receiving vardenafil (20 mg) successfully reported completing intercourse, compared with twenty-two percent receiving placebo.²⁸ Some of our patients initially use one of these medications with varying results but of the men who had success with our product none continued to use these medications. A success rate of roughly thirty percent has been noted following radical prostatectomy.³³ We have one patient in this category with no results yet (patient just stated in the program).

CARDIOVASCULAR EFFECTS. No differences were noted between vardenafil and placebo with regard to exercise time or time to first awareness of angina, but vardenafil did significantly prolong the time to ischemic threshold. No negative hemodynamic effects.⁴¹ An investigation of reports of sildenafil associated deaths showed no difference from expected death rates.⁴² It must be recognized that sexual activity itself is associated with a small risk of myocardial infarction,⁴⁴ and cardiovascular assessment should be considered prior to treatment of ED in any patient considered at increased risk for a cardiac event.

Princeton Consensus Panel was convened to review existing data and provide recommendations regarding the treatment of sexual dysfunction in men with heart disease.⁴⁵ Those recommendations indicate the need for no additional evaluation prior to treatment for men in a low-risk group, including those with controlled hypertension; mind, stable angina; history of uncomplicated myocardial infarction, and mild valvular disease. A high risk group was identified in whom treatment of sexual dysfunction should be withheld until further safety data could be accumulated. This group included men with unstable or refractory angina, uncontrolled hypertension, high-grade congestive heart failure, myocardial infarction within the previous 2 weeks, high risk arrhythmias, obstructive cardiomyopathy and moderate to severe valvular disease. Men with intermediate risk, eg, those with moderate angina or recent myocardial infarction (<6 weeks), should undergo further cardiac evaluation before restratification into one of the other groups.

RISKS WITH PHARMALOGICAL TREATMENT. The single important contraindication is the use of any nitrates, either on a chronic or intermittent basis, due to the potential for significant hypotension. The most common adverse effects are headache (15%), flushing (10%, nasal/sinus congestion (8%), dyspepsia (7%), and transient color vision changes (3%).^{3,27,29} No differences were noted between vardenafil and placebo with regard to exercise time or time to first awareness of angina, but vardenafil did significantly prolong the time to ischemic threshold. No negatives hemodynamic effects.⁴¹ No difference from expected death rates.⁴² It must be recognized that sexual activity itself is associated with a small risk of myocardial infarction,⁴⁴ and cardiovascular assessment should be considered prior to treatment of ED in any patient considered at increased risk for a cardiac event.

(In my study the only side effect that we were aware of was an allergic reaction to the skin. The patient had itching with no rash. It was not clear which compound of our product (cream, progesterone, or testosterone), caused the itching but it was very easy corrected with an antihistamine and the patient continued to take the product with great success.)

OTHER TREATMENT OPTIONS FOR ED. Penile injections with vasoactive medications are effective in 70% to 80% of patients have an onset of action within 10 minutes and are nearly painless.^{46,47} Alprostadil^{48,49} is most frequently prescribed but can cause an unpleasant burning sensation in about 20% of men. Papaverine and phentolamine can be used to avoid this problem or used in combination with alprostadil for greater efficancy.⁵⁰ In a study of 615 cases of men using penile injection therapy, penile fibrosis noted in 3% and 4% of men experienced a prolonged erection, representing 0.3% of injections.⁴⁷

Intraurethral suppositories of alprostadil avoid penile injection but are less effective and require the use of a tourniquet at the base of the penis for optimal results.⁵³ Initial treatment should occur in a health care environment with proper monitoring due to the rare occurrence of syncope.

Surgical implants remain a highly successful and satisfying treatment for men whose condition has failed oral therapy and find other treatment options unsatisfactory.^{55,56} The primary risks are device failure (2% at 2 years; 14% at 5 years) and infection in 2% to 3% of cases.^{55,56,58}

OTHER ORAL THERAPIES. Yohimbine is a plant-derived a-adrenergic inhibitor with limited efficacy in the treatment of ED.⁶⁰ Despite aggressive marketing, no data support the assertion that nutritional supplements, herbal therapies or vita have any beneficial effect in the treatment of ED.¹ (See appendix for other therapies.)

HYPOGONADISM. A man with symptoms such as diminished libido and ED in association with a low serum testosterone level, the condition is termed hypogonadism.²¹ Other symptoms and signs of hypogonadism include depressed mood; reduced energy, muscle mass and strength; reduced bone density; anemia; fatigue; and impaired cognition. Less well-recognized sexual symptoms of hypogonadism include difficulty achieving orgasm, diminished intensity of the orgasm, reduced sexual sensation in the penis and reduced ejaculate volume.²¹

Hypogonadism is quite common, since testosterone levels decline 1% per year beginning around 40 years of age.^{61,62} A major issue for clinicians is whether to first treat his ED, his hypogonadism, or both in combination. Treatment of hypogonadism results in reliable improvement in the symptoms of diminished libido and feelings of enhanced sexuality.^{63,64} However, ED itself may not respond as well, particularly in older men, due to coexisting vascular pathology.

FORMS OF TESTOSTERONE SUPPLEMENTATION. Gels have become the favored mode of treatment for many patients due to their high efficacy in restoring physiological testosterone levels.⁶⁵ Oral agents available in the United States all share a significant risk of hepatotoxity.⁶⁷ An informal survey of Boston pharmacies in Apr. 2004 revealed a monthly treatment cost of approximately $220 for gels and $24 for injections.

RISKS OF TESTOSTERONE TREATMENT. Risks include erythrocytosis in as many as 50% of men receiving injections, but in only 5% of men using gels or patches.²¹ Gynecomastia, peripheral edema, exacerbation or de novo sleep apnea, acne and mild weight gain occur in less than 2% of men.⁶⁴ Testicular atrophy can occur, more prominently in younger men. In addition, men must be advised that fertility will be impaired while receiving exogenous testosterone due to negative feedback on pituitary gonadotrophins.⁶⁸ Transdermal preparations are associated with local skin reactions in 3% to 5% of men using gels and as ma y as 40% of men using patches.²¹ There is no evidence that testosterone supplementation represents a risk for cardiovascular disease; on the contrary, some studies suggest that it may even be beneficial.^{69,71} Although testosterone treatment may reduce high-density lipoprotein cholesterol, total cholesterol is generally reduced as well, resulting in a neutral net effect.⁷² Liver toxicity does not seem to be associated with Transdermal or intramuscular preparations of testosterone.²¹

TESTOSTERONE AND THE PROSTATE. The greatest concern of clinicians regarding testosterone replacement therapy (TRT) is possible stimulation of an occult prostate cancer. This follows from the work of Higgins et al in the 1940s,⁷³ which showed that prostate cancer was androgen-sensitive by following chemical markers in an uncontrolled study of 8 men with metastic prostate cancer who underwent bilateral orchiectomy.

### Research Study on Men's Erectile Dysfunction Using Bio-Identical Progressive/Testosterone Cream vs Pharmaceuticals

### BACKGROUND SUMMARY

My study was designed to evaluate the effect of bio-identical progesterone/testosterone on the erectile dysfunction of men. The study was performed over a period of two years. Twenty patients were randomly involved in this clinical study ranging in ages from twenty-one to eighty-eight years. The men had a multiplicity of medical conditions including one or more of the following disease processes: hypertension, BPH, diabetes, elevated cholesterol, cardiovascular disease, lung or prostate cancer, cerebral palsy, COPD, renal insufficiency and depression. At the time this study was undertaken the initial goal was to improve erectile dysfunction regardless of the preexisting medical problems. At first, I attempted to distinguish between organic and psychogenic erectile dysfunction. I attempted to optimize their medical management and simply added our product to there daily regiment without altering any other variables.

My formulation is a Bio-Identical Progesterone-Testosterone compound in a cream or gel. The men in the study received a small amount of this mixture daily. They used one to three pea sized allocates of cream, to their inner arms, chest, face, or neck.

Findings:

We have found in our study that men with ERECTILE DYSFUNCTION have a tendency to have low free testosterone, low progesterone, and a mid/normal to high estrodiol. Our initial goal in this study was simply to correct the hormone imbalance in our patients by replacing the hormones that were deficient. Knowing that hormone imbalance is one component of ERECTILE DYSFUNCTION, we were only hopeful that correcting this one component would result in full erections. To our great surprise we had tremendous success in correcting ERECTILE DYSFUNCTION despite the number of co-existing medical problems. In our study ERECTILE DYSFUNCTION showed significant improvement in as little as three weeks and as long as six months with an average being two to three months. Of the twenty patients in the study, twelve had low to low normal progesterone, twelve had low to low normal free testosterone, 13 had mid/normal to high estrodiol, and all of the men except for one, started out with a PSA in normal range. All of the patients in the study were evaluated for the origin of their ERECTILE DYSFUNCTION prior to entering the study. None of the patients, based on the questionnaire and exam, gave any evidence of significant psychogenic ERECTILE DYSFUNCTION. All of the men in the study were having difficulty obtaining a full erection prior to starting the study and most of the men were only able to obtain a partial erection under any circumstances, i.e. sleep, looking at photographs, thinking about the process, involved in the process, or with stimulation by their partner.

### Below are the Findings by Individual Patients in the Study

1) DA—85—male—history of hypertension, hypercholesterolemia, and BENIGN PROSTATE HYPERTROPHY—At the beginning of the study, the patient reported to having no erection to a soft/partial erection. After two months of using the cream, the patient developed a full erection. Although the patient had significantly good results, he stopped using the cream, noting that he was too old and did not have a partner. The patients PSA was in the normal range (2.6-3.5) while using the cream. Six months later, he had an elevated PSA of 5.0. Evaluation of the elevated PSA did not reveal any evidence of cancer, and I do not feel the elevated PSA was related to the Progesterone/Testosterone regimen.
2) SC—73—male—history of hypertension and BENIGN PROSTATE HYPERTROPHY—At the time the patient started the cream he was not able to obtain an erection. After three months he was having a full erection. Patient's PSA went from 1.9 up to 5.7 after four months of using the cream. We stopped the cream, repeated the PSA four months later and it remained stable at 5.6, repeated the PSA in another four months and it had risen to 7.0. Urological evaluation of the patient has revealed no evidence of cancer and the PSA has remained elevated seven months after stopping the Progesterone/Testosterone cream.
3) CW—70—male—history of hypertension, non-insulin dependent diabetes, and BENIGN PROSTATE HYPERTROPHY—After six months the patient showed significant improvement, but was still supplementing the cream with oral phosphodiesterase type 5 inhibitor. One year later on the cream, the patient was having a full erection without the oral phosphodiesterase type 5 inhibitor. During the study the patients PSA remained unchanged.
4) DL—21—male—history of cerebral palsy and hypertension—In a three to six month period the patient had improvement in his ERECTILE DYSFUNCTION back to a place of normality. The corresponding erection showed a free testosterone that went from 72 to 168 and his estrodiol went from 25 to 14. Both of these would be desired effects of the Progesterone/Testosterone cream.
5) NW—85—male—history of hypertension, non-insulin dependent diabetes, gout, hypercholesterolemia, renal insufficiency, and prostate cancer—Patient started the study with a very low free testosterone and a mid/normal estrodiol with a PSA of 5.5. The patient used the cream for two months, stopped using it and noted that he was not having any success and was not willing to continue use any longer. But of note, over a period of many months his PSA has gone from 7.7 down to 0.7.
6) LR—80—male—history of hypertension, atrial fibrillation, BENIGN PROSTATE HYPERTROPHY, cardiomegaly, hypercholesterolemia—Patient was able to obtain a full erection after two months of using the cream. Of note, he lost ten pounds, largely from his abdomen after being on the cream for four months. He stopped the cream and insisted that he had cancer. We explained to the patient that progesterone cream often causes a person to loose some abdominal girth. Several months after stopping the cream, the patient regained most of the weight he had previously lost.
7) WW—55—male—history of hypertension, status post MI, and depression—Patient stopped using the cream after two months. He noted that it was not working. Of note, he did have the characteristics of the average patient with ERECTILE DYSFUNCTION, low free testosterone and progesterone and a mid/normal to high estrodiol. We feel the patient would have had success had he continued in the study.
8) RJ—51—male—CVA, seizure disorder, alcohol abuse—Patient obtained a full erection after two months of using the cream, but noted to still need the help of Viagra, which I feel was probably psychogenic. Patient was not able to obtain an erection prior to the cream despite the use of Viagra.
9) DM—66—male—history of CHRONIC OBSTRUCTIVE PULMONARY DISEASE, BENIGN PROSTATE HYPERTROPHY, and non-insulin dependent diabetes—Very weak to partial erection to a full erection in three months. Patient started off with an elevation in his PSA of 6.1 and it subsequently went down to normal during the study. Also note the patient was started on a natural product for prostate health during the study.
10) RS—56—male—history of hypertension and non-insulin dependent diabetes—Patient initially had no erection without an oral phosphodiesterase type 5 inhibitor. After five months of using the cream, patient was able to get a full erection without an oral phosphodiesterase type 5 inhibitor.
11) SG—74—male—history of lung cancer, hypertension, non-insulin dependent diabetes, and renal insufficiencies—Patient just started in the study, but has the same characteristic hormone imbalance that we are commonly seeing with patients with ERECTILE DYSFUNCTION.
12) WJ—68—male—history of hypertension, hypercholesterolemia, BENIGN PROSTATE HYPERTROPHY, renal insufficiencies and rectal cancer—Patient had minimal response after using the cream for two months and stopped. He also had the characteristic low progesterone and testosterone with the high estrodiol. We feel that if the patient gives the regimen more time he will have success.
13) GH—46—male—history of hypertension—Two to three weeks on the cream the patient was having a full erection. Question psychogenic component to his ERECTILE DYSFUNCTION.
14) LA—78—male—history of CHRONIC OBSTRUCTIVE PULMONARY DISEASE, CORONARY ARTERY DISEASE, hypertension, renal insufficiency and non-insulin dependent diabetes—Patient initially had no response after three months of using the cream. We increased the amount that the patient was using and over the next three months he was able to regain a full erection.
15) HV—79—male—history of hypertension, hypercholesterolemia, and a GASTROINTESTINAL malignancy—Patient had not had a full erection in over three and a half years. Two and a half months after starting on the cream was able to obtain a full erection. He too had the characteristic pattern of patients with ERECTILE DYSFUNCTION.
16) HS—77—male—history of hypertension, CORONARY ARTERY DISEASE, CONGESTIVE HEART FAILURE, CHRONIC OBSTRUCTIVE PULMONARY DISEASE, hypothyroidism—Patient was able to obtain a full erection after one month. Patient did have some itching associated with use of the cream, but no observable rash. After adding an antihistamine to his regimen his itching resolved.
17) SE—88—male—history of prostate cancer, anemia, BENIGN PROSTATE HYPERTROPHY, non-insulin dependent diabetes—Patient's hormone profile showed extremely low progesterone, free testosterone that was non detectable, and an estrodiol that was low along with a PSA of less than 0.1. Patient was receiving hormonal treatment for his prostate cancer and did not want to undergo treatment with the Progesterone/Testosterone cream.
18) LL—52—male—history of hypertension—Patient had minimal response after two months of using the cream and stopped using it, but started the study with the characteristic hormone imbalance we find in men with ERECTILE DYSFUNCTION. Had he been willing to continue with the cream, we feel he would have had good success.
19) JF—49—male—history of hypertension and obesity—Patient has recently started in the study with the same hormonal characteristic pattern we find in patients with ERECTILE DYSFUNCTION.
20) LH—39—male—history of hypertension, and mild renal insufficiency—Patient had two months of use with some improvement without a full erection, but was having no erection prior to using the cream.

What is claimed is:

1) Progesterone/Testosterone Cream to be very helpful in the alleviation of ERCTILE DYSFUNCTION.

2) Effectiveness in helping ERECTILE DYSFUNCTION with progesterone solutions from 0.5 to 5% and testosterone solutions from 2 to 20%, working synergistically in helping ERECTILE DYSFUNCTION.

3) The combination of Progesterone/Testosterone Cream can be helpful in increasing the libido in men.

4) The Progesterone/Testosterone Cream mixture we are using is readily absorbed and does not have to pass through the liver, alleviating the side-effects from the metabolite.

5) After use of the Progesterone/Testosterone Cream for as little as three weeks, men who were previously unable to get full erections have been able to obtain full erections.

6) Men are able to obtain normal erectile function after using the Progesterone/festosterone Cream.

7) We are able to raise their progesterone and free testosterone levels to normal range using Progesterone/Testosterone Cream.
